(19) United States
(12) Patent Application Publication
(10) Pub. No.: US 2018/0173849 A1
(43) Pub. Date: Jun. 21, 2018

(54) COMPUTER-IMPLEMENTED METHOD FOR DESIGNING SYNTHETIC DNA, AND TERMINAL, SYSTEM AND COMPUTER-READABLE MEDIUM FOR THE SAME

(71) Applicant: TUPAC BIO INC., Wilmington, DE (US)

(72) Inventor: Eli LYONS, Shinagawa-ku (JP)

(73) Assignee: TUPAC BIO INC., Wilmington, DE (US)

(21) Appl. No.: 15/849,731

(22) Filed: Dec. 21, 2017

Related U.S. Application Data
(63) Continuation of application No. PCT/US16/69053, filed on Dec. 28, 2016.
Application No. PCT/US16/69053 is a continuation of application No. PCT/US16/39115, filed on Jun. 23, 2016.
(60) Provisional application No. 62/183,362, filed on Jun. 23, 2015.

(51) Int. Cl. G06F 19/28; G06F 17/30; G16H 10/40; G06K 19/06

(52) U.S. Cl. CPC G06F 19/28; G06K 19/06028; G16H 10/40; G06F 17/30879

(57) ABSTRACT

A method, comprising displaying indicia, broadcasting data, or transmitting instructions, to solicit collection of or to access data uniquely representative of, or uniquely indicating, one or more digitally-input synthetic DNA design parameters; establishing direct or indirect communication access and linkage between the terminal and either (a) a store at the terminal, or (b) the at least one remote computer(s), on which are stored, or by which access is available to receive:
data representative of an entire synthetic DNA sequence of a DNA library, the synthetic DNA sequence being (remotely or at the terminal) calculated based on the one or more DNA design parameters; and
data representative of an entire synthetic DNA barcode sequence that is calculated based on the calculated synthetic DNA sequence. A terminal, system and computer-readable medium are also provided.

### TECHNICAL FIELD

The present disclosure generally relates to the design of synthetic DNA. More particularly, the present disclosure relates to a method of designing one or more DNA libraries for one or more DNA strands.

### BACKGROUND

Synthetic DNA is useful for various applications such as genome engineering, functional genomics, bio-detection, drug design, and data storage. Many downstream applications of synthetic DNA require DNA sequencing and subsequent related digital-computer-generated analysis and communication to humans of what are stored in the synthetic DNA strands. However, conventional DNA sequencing often requires homogeneous mixtures of DNA and alignment of short DNA sequencing reads to determine a consensus sequence of the original DNA. This limitation makes sequencing of heterogeneous DNA mixtures difficult and sequencing of longer strands challenging.

As an example, the need to create myriad bacterial clones for screening and full sequencing of potentially-antigen-binding synthetic DNA antibodies is exemplary of the difficulties, at least in terms of this need's drain on laboratory, sequencing and related computing analytical resources.

A related, daunting problem is that DNA sequencing, analysis and display into a format that is understandable to humans, requires extremely large amounts of computing resources to process and interpret the enormous data stored in DNA libraries.

For example, the amount of computing resources required to sequence each unique strand (or quasi-unique, e.g., unamplified strand(s)), such as the amount of one or more of data processing hardware (e.g., non-limiting examples such as processors and storage resources) and data processing network resources required to process each strand(s) during most sequencing methods, is vast. It is estimated that approximately 1 gram of DNA can potentially store up to approximately 700 terabytes of digital data. Another estimate of the vast amount of data storable in DNA approximates that the entire repository of world knowledge stored in thousands of digital data centers and localized databases could all be stored in DNA that would all entirely fit into just one sport utility vehicle (SUV). Therefore, it would be desirable to provide a method of designing synthetic DNA that dramatically reduces the laboratory and computing resources needed to, one or more of (a) identify DNA by sequencing, (b) digitally analyse the resulting data and (c) represent that data in a format that can be understood by humans, more efficiently.

### SUMMARY

First and second aspects of the disclosure may or may not be directed to one or more of respective method and computer-readable medium causing operations, for each comprising any one or more of: displaying indicia, broadcasting data, or transmitting instructions, to solicit collection of or to access data uniquely representative of, or uniquely indicating, one or more digitally-input synthetic DNA design parameters; and establishing direct or indirect communication access and linkage between the terminal and either (a) a store at the terminal, or (b) the at least one remote computer(s), on which are stored, or by which access is available to receive: data representative of an entire synthetic DNA sequence of a DNA library, the synthetic DNA sequence being (remotely or at the terminal) calculated based on the one or more DNA design parameters; and data representative of an entire synthetic DNA barcode sequence that is calculated based on the calculated synthetic DNA sequence. A terminal, system and computer-readable medium are also provided.

Third and fourth aspects of the disclosure may or may not be directed to one or more of respective processor-based terminal and processor-based system, each comprising any one or more of: displaying indicia, broadcasting data, or transmitting instructions, to solicit collection of or to access data uniquely representative of, or uniquely indicating, one or more digitally-input synthetic DNA design parameters; and establishing direct or indirect communication access and linkage between the terminal and either (a) a store at the terminal, or (b) the at least one remote computer(s), on which are stored, or by which access is available to receive: data representative of an entire synthetic DNA sequence of a DNA library, the synthetic DNA sequence being (remotely or at the terminal) calculated based on the one or more DNA design parameters; and data representative of an entire synthetic DNA barcode sequence that is calculated based on the calculated synthetic DNA sequence. A terminal, system and computer-readable medium are also provided.

Additional or alternative aspects of the disclosure are found in the appended claims. Further aspects, embodiments, features, and advantages of the embodiments, as well as the structure and operation of various embodiments are described in detail below with reference to accompanying drawings.

### BRIEF DESCRIPTION OF THE DRAWINGS

In the accompanying drawings, which form a part of the specification and are to be read in conjunction therewith, and in which like reference numerals are used to indicate like features in the various views:

FIG. 1 is a schematic diagram of three exemplary digitally stored and operated computer network environments, in which embodiments may be implemented;

FIG. 2a is a diagram illustrating a method of designing a synthetic DNA barcode system according to an embodiment;

FIG. 2b is a diagram illustrating a method of designing a synthetic DNA barcode system according to an embodiment;

FIG. 3 is a diagram illustrating a method of designing a synthetic DNA barcode system according to an embodiment;

FIG. 4 is a diagram illustrating a method of designing a synthetic DNA barcode system according to an embodiment;

FIG. 5 is a diagram illustrating a method of designing a synthetic DNA barcode system according to an embodiment;

FIG. 6 is a diagram illustrating a method of designing a synthetic DNA barcode system according to an embodiment;

FIG. 7 illustrates a graphical user interface of a synthetic DNA barcode design system according to an embodiment;

FIG. 8 illustrates a graphical user interface of a synthetic DNA barcode design system according to an embodiment;

FIG. 9 illustrates a graphical user interface of a synthetic DNA barcode design system according to an embodiment;

FIG. 10 illustrates an exemplary file for setting probabilities for a synthetic DNA barcode design system according to an embodiment;

FIG. 11 is a flow diagram for an algorithm for a barcode sequence design according to an embodiment;

FIG. 12 is a flow diagram for an algorithm for a randomized region design, according to an embodiment;

FIG. 13 is a flow diagram for an algorithm for a synthetic DNA barcode design system, according to an embodiment;

FIG. 14 illustrates an example computer useful for implementing components of the embodiments, along with several network features that may be used in embodiments; and

FIG. 15 illustrates an exemplary embodiment of the computer of FIG. 14 in a mobile terminal.

### DETAILED DESCRIPTION

In one aspect, embodiments of the disclosure are concerned with one or more of requesting, receiving, storing and transmitting data to design and create a digital representation of (a) at least one synthetic DNA strand and (b) one or more associated synthetic DNA barcodes, each of which DNA barcode (or DNA barcode set) being uniquely indicative of the at least one synthetic DNA strands.

In embodiments, at least one digitally represented copy of each designed synthetic DNA is stored digitally, or otherwise archived, so it need not be sequenced—because it is already known. As such, in embodiments, only the designed and associated synthetic barcode(s) need be sequenced to determine the identity of the known and archived synthetic DNA sequence strand, partial gene, gene, chromosome or genome.

In embodiments, since the barcode sequences are known, an entire genome can be identified by searching a digital database containing the synthetic genome design. In the case, one or more barcodes may be created and used, allowing the mixing and matching of synthetic DNA subparts to create various DNA sequence combinations.

In certain cases, embodiments are concerned with one or more of the solicitation of parameters, receipt of data representing parameters, calculation of synthetic DNA strand(s), calculation of DNA barcode(s) that are automatically associated with the calculated strands, and transmission of data representative of those synthetic DNA and DNA barcode(s) in digital form to at least one remote computer(s) (e.g., but not limited to, any combination of one or more of, a bank of, and geographically disparate communicatively connected, server(s) that manipulate user data). Ultimately, though not in certain method, terminal, system and computer-readable media embodiments, these digitally represented synthetic DNA and associated DNA barcodes (and related digital instructions to analyse or sequence them), may or may not also be executed at one or more server(s), storage device(s) or other computer hardware holding or capable of selectively displaying data set(s) being operated on.

The data and digital data processing resources required on a user terminal and other components of a DNA data digital computer network are therefore reduced by one or more of (1) reducing or completely eliminating the number of short DNA reads when sequencing entire DNA strands because the computer pre-designed DNA strands are already known and pre-labelled/associated with computer designed synthetic DNA labels, (2) preventing superfluous data from being transmitted to or stored on one or more storage devices or data structures, and (3) restricting unneeded data transmission and computation through the network and therefore receipt and computation at one or more terminals in the DNA data digital computer network.

It is apparent that the benefits of widespread DNA data analysis and efficient use of limited digital computing resources, seemingly cannot coexist, creating a technically derived tension. For example, one genome of one human organism requires at least about 700 megabytes of digital storage resources. Yet, thousands of DNA strands are being sequenced every year, with ever increasing acceleration of numbers that are sequenced. When using unknown natural sequences, the computing resources associated with piecemeal high throughput DNA sequencing reads of many pieces of a DNA strands, is astounding. The unprecedented expected deluge of DNA information that needs both sequencing and digital computer analysis can be attributed to the technical efficiencies provided by the shear amount of DNA data available and desirable for sequencing.

In embodiments, certain of the below-indicated non-limiting technical advantages and/or others, each of which depend upon what particular combination of features disclosed herein is found in an embodiment, are realized only upon persistent and arduous study through both (a) discovering the very existence of the above-indicated technical tension, and (b) inventing the technical solutions disclosed in part herein.

In embodiments, resulting parameter request, synthetic DNA calculation, data transmission, and use of (without need to sequence) synthetic DNA having pre-associated barcodes to represent these pre-determined, and already archived DNA libraries, thereby frees up processing resources both at an individual level such as at a terminal, and all the more so collectively, across even a global network of data storage and processing infrastructure facilitating the ebb and flow of DNA data of millions, if not even billions, of DNA data computer network. These embodiments quite un-expectantly provide the unpredictable result(s) of reducing both sequencing and computing resources regarding data transmitted to and from various sequencing machines, terminals and other computers (and databases) communicatively connected to one or more digitally stored and operated digital computer networks.

In embodiments, operations by which data set transformations are made improve data security by preserving the secrecy of certain DNA sequence data through limited access to the sequences. For example, synthetic DNA sequence data may or may not be stored exclusively at a pharmaceutical company's server(s).

In embodiments, operations by which data set transformations are made increase system operational efficiency at each terminal.

In embodiments, operations by which data set transformations are made strike an optimal balance between improving data security by preserving synthetic DNA sequence secrecy on the one hand, and increasing system operational efficiency, on the other hand, all the while allowing continued data (via associated DNA labels) exchange and aggregation between one or more user computers communicatively connected to at least one digitally stored and operated computer network.

In embodiments, operations by which data set transformations are made reduce the demands on sequencing equipment.

In embodiments, operations by which data set transformations are made reduce error introduced during sequencing and related laboratory and computational analysis.

In embodiments, operations by which data set transformations are made provide at least a 10 fold increase in heterogeneous high throughput sequencing speeds. Scientists that wish to sequence heterogeneous DNA mixtures of DNA using short read sequencing such as manufactured by Illumine® technology, need first to isolate the DNA samples, which requires laborious steps and decreases throughput.

In embodiments, typically during protein interaction screens, such as in-vitro fragment antibody screens using phage display, there is a heterogeneous mixture of phages obtained from the screen that contain the DNA of interest. Therefore, technicians must take steps such as bacterial cloning to isolate each DNA sample and then pick these bacterial clones individually before sequencing. This procedure requires manual steps, and throughput is on the order of 1,000-10,000 screened unique candidate sequences. However, because these steps are eliminated in certain embodiments, then the throughput is increased by 10 times these amounts and, in embodiments, the bias that these steps introduce into experiments can be eliminated as well.

In embodiments, one or more of the efficiencies disclosed herein are realized by allowing for the instantaneous design of, for example, 100 variations of each peptide and each antibody coding sequence.

In embodiments, a synthetic barcode comprises a synthesized segment of DNA that does not code for a peptide, protein or act as a functional binding site, but instead stores data as a synthetic DNA sequence. In addition, the data stored as a synthetic DNA sequence is a code that identifies a larger region of synthetic DNA.

In alternate embodiments, a synthetic DNA barcode comprises a synthesized segment of DNA that codes for any type of information, optionally any type of digital information and is on the same DNA strand that contains regions of DNA that do in fact have and/or code for biological functionality, such as for coding for a protein, a micro RNA, or other biologically functional component.

In embodiments, a barcode is a data container which can hold any data that a user wishes to associate with an entire synthetic DNA/polynucleotide design. For example, barcodes may represent photographic pictures or other documents converted into DNA sequences (which are optionally then rearranged in accordance with a cipher that encrypts the DNA sequences) that are stored as one or more synthetic DNA barcode(s). These bar codes do not code for proteins and are part of the same strand(s) as a larger synthetic DNA design.

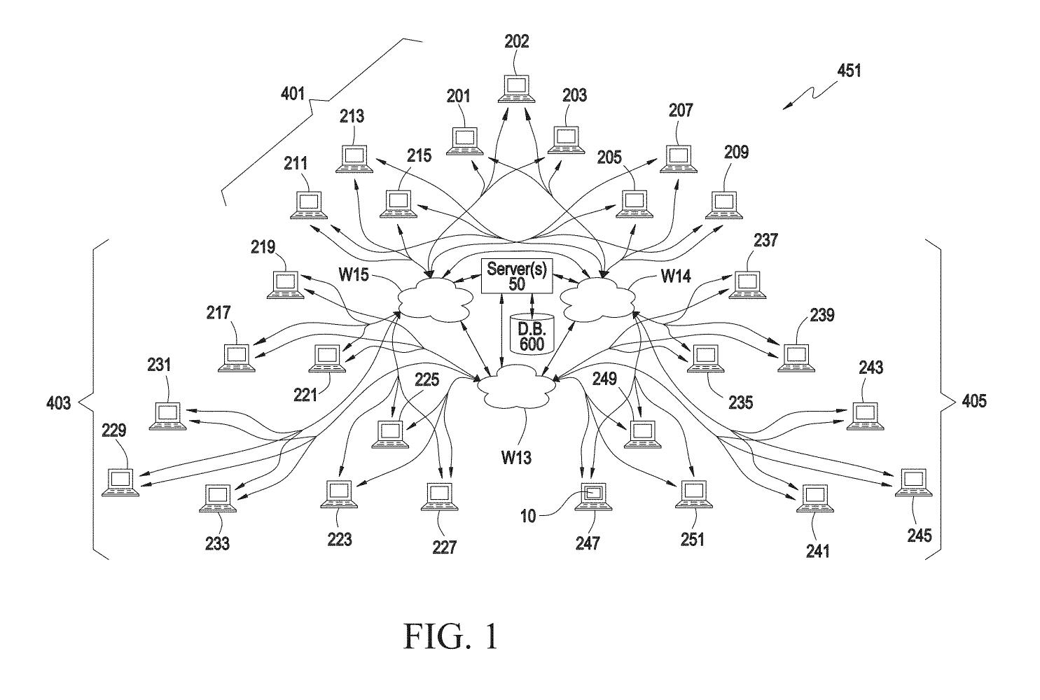

As illustrated in FIG. 1, in embodiments, terminal and/or system data flow configurations selectively solicit synthetic DNA sequence parameters, and one or more of (a) store and (b) transmit data representative of synthetic DNA sequences calculated based on the parameters, with an unexpected technical efficiency not previously seen, in view of the above-indicated seemingly irreconcilable tensions.

In embodiments, server(s) 50 execute instructions for calculating one or more synthetic DNA sequence(s) and synthetic DNA barcode(s) based on predetermined, previously stored, or previously received data representative of synthetic DNA.

In embodiments, at least one of terminals 201 to 251 transmit instructions to server(s) 50 to execute instructions causing successful or unsuccessful creation of one or more of (a) data representing a synthetic DNA sequence(s) and (b) data representing one or more uniquely associated and uniquely represented synthetic DNA barcode(s), both of which are stored. One or more portions of the synthetic DNA barcode(s) are optionally transmitted to a sequencer or associated computing devices that assist in the many reads required during high-throughput heterogeneous DNA strand sequencing. In turn, terminals, such as ones more directly associated with high throughput heterogeneous DNA sequencers, may (or may not) not need the computational resources typically needed during the myriad reads required during high throughput heterogeneous sequencing, thereby creating an acceleration and scaling of at least several of the technical advantages of various herein disclosed embodiments, for example, across the entirety of network 451.

In embodiments, calculated synthetic DNA sequences may (or may not) be exclusively stored at server(s) 50, or transmitted only once (or optionally only as needed) to one or more terminals. This feature, wherein the synthesized DNA sequences are potentially, always stored only in a single centralized location, reduces the risk of data breach and hacking.

In embodiments, associated calculated synthetic DNA barcodes may (or may not) be exclusively transmitted to one or more terminals, such that the calculated DNA sequences are never transmitted to any other of the one or more terminals.

The following describes a system for designing synthetic DNA strand(s) and correlated, and uniquely and individually-associated DNA barcodes, according to embodiments illustrated in FIGS. 1-15. FIG. 1 is a diagram of computer network 451 including three exemplary environments, in which embodiments may be implemented. While the following is described in terms of FIG. 1, the embodiments are not limited to the environment(s) illustrated in FIG. 1. For example, any system having generally the structure of FIG. 1, or that would benefit from the operation, methods and functions as described herein may be used.

In exemplary embodiments, system 451 shows terminal clients 201-251 each or collectively comprising one or more browser(s) 10 of terminal 247 (browser also in each of other terminals, but not shown), which is/are used to connect to server(s) 50 over one or more networks W13, W14, and W15.

In embodiments, one or more of these terminals may or may not be communicatively connected to one or more DNA sequencer(s).

In embodiments, one or more of these terminals may be communicatively connected to one or more DNA sequence database(s).

According to embodiments, browser 10 may include any device, application or module that enables a user or computer to navigate and/or retrieve data from another data source, typically over a network. Browser 10 may include any conventional web browser such as those that are widely available. According to further embodiments, browser 10 may also be configured to use any number of protocols, known now or developed in the future, including protocols such as HTTP, FTP, and underlying protocols such as TCP/IP or UDP. In embodiments, browser 10 is configured to run (or execute) web applications without a GUI as a headless browser. Web applications are applications that can be hosted within a web browser or those that can be accessed, for example, over a network such as the Internet or an intranet.

Browser 10 can further communicate with an input (not shown) to allow a user to input data, to input commands, or to provide other control information to browser 10. Browser 10 may request content from one or more server(s) 50, based on prior user input that is stored at one or more terminal(s) or server(s) 50 before accessing server(s) 50, and upon which instructions later sent to server 50 are calculated. Server(s) 50 may respond to the request by providing content back to browser 10 and client 201 via network W13. Browser 10 may also be configured to retrieve content from server(s) 50 without user intervention.

In embodiments, network(s) W13, W14, and W15 can be any type of data network or combination of data networks including, but not limited to, a local area network (LAN) accessed locally or remotely such as via a VPN, a medium area network, or a wide area network such as the Internet. Network W13, for example, can be a wired or wireless network that allows client terminal 247 and server(s) 50 to communicate with each other. Network W13 can further support world-wide-web (e.g., Internet) protocols and services.

Server(s) 50 provides content (e.g., web pages, applications (or “apps”), audio, video, etc.) that can be retrieved by client terminal 247 over network W13. Content retrieved by client 247 can be disseminated via browser 10. In various embodiments, server(s) 50 and/or browser 10 includes one or more features of content manager 200, which is described further below.

In embodiments, a base functional component of one aspect of the disclosure is composed of at least one of a plurality of terminals 201 to 251, configured to be ordered by predetermined default settings or user-selected settings and/or software instructions into one or more dynamically changing and rearranging user terminal groupings. Certain network terminals and/or systems, e.g., system 451, connect and allow exchange of information between local or far flung terminals within and from at least, but not limited to, three distinct types of networks W13, W14, and W15.

In embodiments, terminal group 401 comprises terminals 201 to 215, terminal group 403 comprises terminals 217 to 233, and terminal group 405 comprises terminals 235 to 251, each group and collective groups illustrating flow of data, albeit on a very small scale, among and across varied networks, such as clear network W13, darknet or darkweb W14 (e.g., employed via The Onion Router (Tor)), and peer-to-peer network

W15 via at least one (or more) server(s) 50. Server(s) 50 receive, store, retrieve and deliver, across and at numerous and geographically disparate locations, user account data on one or more databases 600.

In embodiments, terminal and system operations may or may not in whole or in part be effectuated, executed, or implemented on or via clear network W13 (comprising at least all of, or just a portion of, terminal groups 403 and 405) whereby individual terminals, server(s) 50, or a combination thereof, calculate the actions to be taken on respective data sets, and propagate(s) those actions out to the network via server(s) 50 and beyond to all other users.

In embodiments, terminal and system operations may or may not in whole or in part be effectuated, executed, or implemented on or via dark net W14 (comprising at least all of, or just a portion of, terminal groups 401 and 405) whereby individual terminals, server(s) 50, or a combination thereof, calculate the actions to be taken on respective data sets, and propagate(s) those actions out to the network via server(s) 50 and optionally beyond to other users.

In embodiments, terminal and system operations may or may not in whole or in part be effectuated, executed, or implemented on or via a peer-to-peer network W15 (comprising at least all of, or just a portion of, terminal groups 401 and 403) whereby one or more terminals, server(s) 50, or a combination thereof, calculate the actions to be taken on respective data sets, and propagate(s) those actions out to the network.

In embodiments, each terminal may or may not be geographically remote from or local to the computers that access server(s) 50.

In embodiments, each terminal may or may not be part of one or more device set(s), the one or more device set(s) that may or may not comprising only one or multiple-single user, entity (e.g., informal group) or participant-controlled, owned or used device(s).

In embodiments, any one or more of these terminal(s) or device set(s) may or may not include for example remote log-on and/or remote usage via any Web-capable device to a Web-based ASP or peer-to-peer decentralized network even though device ownership, possession and/or control is only temporary and/or through established via other-user-owned or installed applications, such as by embedded or remote implementation via a widely used social media site application or website.

In embodiments, client terminal 247 and server(s) 50 can each be implemented on a computing device. Such a computing device includes, but is not limited to, a personal computer, mobile device such as a mobile phone, workstation, embedded system, game console, television, set-top box, or any other computing device that can support web browsing. Such a computing device may include, but is not limited to, a device having a processor and memory for executing and storing instructions. Such a computing device may include software, firmware, and hardware. The computing device may also have multiple (one or more) processors and multiple (one or more) shared or separate memory components. Software may include one or more applications and an operating system. Hardware can include, but is not limited to, a processor, memory and graphical user interface display. An optional input device, such as a mouse or touch screen, may be used.

### Software Instructions and Laboratory Methods

Another aspect of the disclosure is a method. Embodiments of the instant disclosure provide a method of designing synthetic DNA libraries and DNA barcodes for synthetic DNA strands that are designed for more efficient DNA sequencing.

In embodiments, a computer-implemented system automates the design of synthetic DNA strands and synthetic DNA barcodes.

In embodiments, a barcode and a DNA strand (e.g., a gene) are synthesized together, simplifying the genetic engineering process, allowing for the production of large synthetic libraries of genes that can be barcoded, shuffled if desired, and/or used in in-vitro and in-vivo experiments.

In embodiments, a barcode is outside the area of a correlated gene, allowing the barcode to be in the genome of a living organism or expression system with minimum effect. For instance, exemplary applications are in-vitro antibody/biologic screens for drug development by use of phage display, CIS display or other biologic screening methods.

In exemplary embodiments, a method is used in conjunction with genetic screens to determine the effect of gene mutations or to optimize the function of a gene. With more particularity, embodiments include one or more of designing novel variations of a gene, manufacturing such designs in synthetic DNA, and using genetic engineering techniques to insert such genes into genes of an organism (such as into a bacteria or yeast) to observe their effect on cellular processes within the organism.

In embodiments, a method of barcoding genes in advance of a high-throughput experimental process without effecting protein expression is provided.

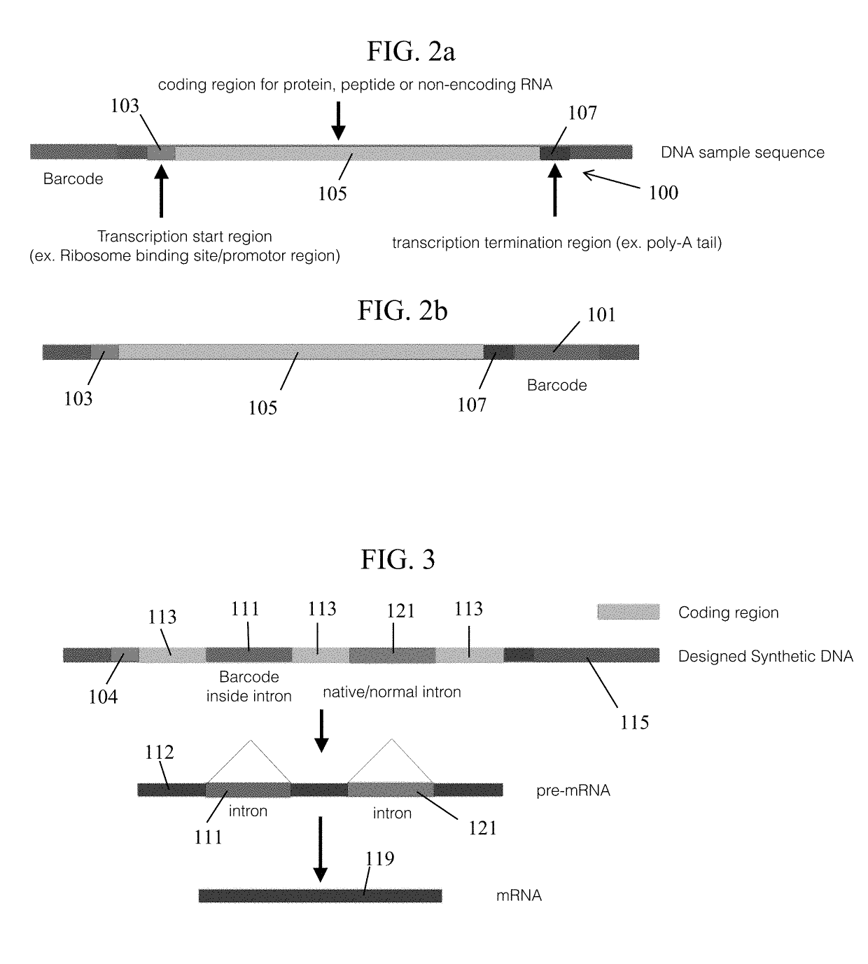

Additional embodiments are described in detail below. In particular and with regard to FIGS. 2a and 2b-FIG. 13, design manager software module 200 provides digital computer program instructions to allow at least one processor and at least one memory to perform any one or more of the steps, sub-steps, routines, and subroutines described herein, such as variously described in diagrams, tables, GUIs and flow charts.

FIG. 2a and FIG. 2b are diagrams illustrating methods of designing a synthetic DNA barcode according to embodiments of the present disclosure.

In embodiments, referring to FIG. 2a, synthetic DNA barcode 101 is not transcribed (or later translated) into ribonucleic acid (RNA) when in contact with molecular biology machinery, (e.g., for at least one particular DNA strand 100 or more generally for any particular gene) such as transcription factors or polymerase. This is achieved by placing the barcode upstream of (a) a transcription start site or region 103, such as at a ribosome binding region, start codon or promoter region, and (b) a coding region 105, as illustrated in FIG. 2a.

In embodiments, synthetic DNA barcode 101 is placed downstream of transcription or translation termination sites or regions such as a poly-A tail 107, as illustrated in FIG. 2b.

In both FIGS. 2a and 2b, the synthetic DNA region 105 neighbouring the barcode may or may not code for, e.g., one or more data storage media and one or more various biological products including, but not limited to, proteins, peptides, and non-encoding RNA.

FIG. 3 is a diagram illustrating a method of designing a synthetic DNA barcode according to embodiments of the present disclosure. In embodiments, synthetic barcode 111 is designed to be positioned inside of a region of a gene that is removed by RNA splicing before messenger RNA (mRNA) transcription and transfer RNA (tRNA) translation, these regions commonly being referred to as introns, as illustrated in FIG. 3. Barcode 111 is positioned within a non-coding region of designed synthetic DNA strand 115. Native/normal intron 121 is also positioned within a non-coding region of designed synthetic DNA strand 115. Yet, coding regions 113 are both sides and between these two introns. Splicing of DNA 115 results in the removal of a section of DNA 115 to form pre-mRNA 112, which still contains barcode 111 and normal intron 121. Then, mRNA 119 is formed by removal of these introns 111 and 121.

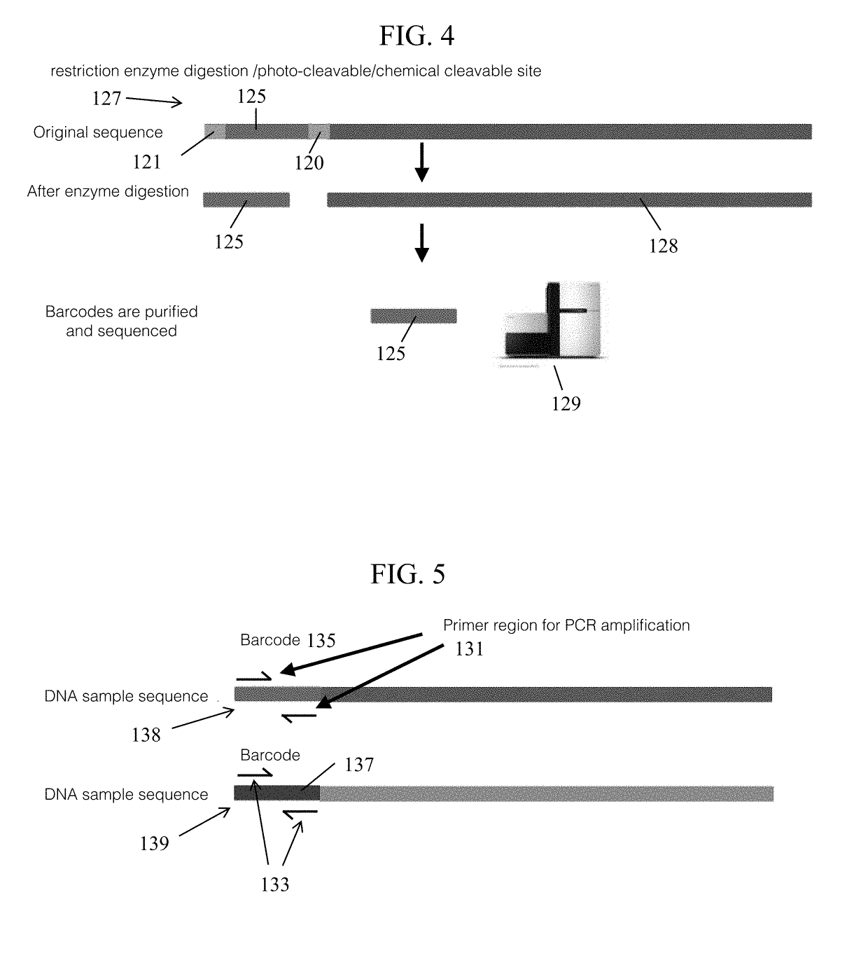

FIG. 4 is a diagram illustrating a method of designing a synthetic DNA barcode according to embodiments of the present disclosure. In embodiments, one or more of restriction enzyme digestion site 120 (and/or a photo-cleavable site, and/or a chemically cleavable site), is included in designed synthetic DNA strand 127. The cleavable site 120 and 121 flanks barcode 125, allowing isolation and purification of synthetic DNA barcodes for sequencing, as illustrated in FIG. 4. Synthetic DNA sequence 127 and barcode 125 result. In embodiments, only bar code 125 is sequenced by sequencer 129.

FIG. 5 is a diagram illustrating a method of designing a synthetic DNA barcode according to embodiments. In embodiments, primer regions 131 and 133 for polymerase chain reaction (PCR) amplification are designed as part of a respective DNA barcodes 135 and 137, specifically to amplify DNA barcodes 135 and 137 (of respective DNA strands 138 and 139) and purify each or multiple barcode(s) for DNA sequencing, as illustrated in FIG. 5.

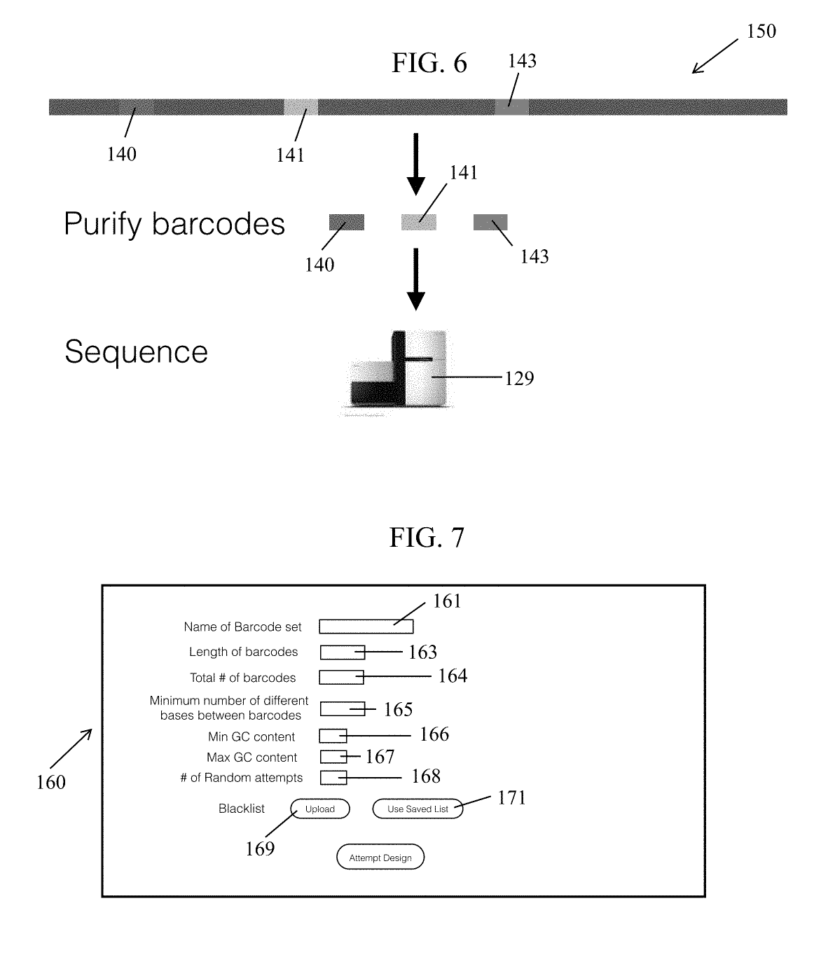

FIG. 6 is a diagram illustrating a method of designing a synthetic DNA barcode according to embodiments. In embodiments, a method is provided to create one or more of a portion of a genome (such as for coding one or more tissue type(s), organ(s) or biological system) and an entire synthetic genome, from which barcodes can be purified and sequenced to determine the rest of a portion or of the entire genome sequence. This in turn provides identification of a complete (already archived) DNA-functional portion or complete synthetic DNA sequence. For example, bar codes 140, 141 and 143 are purified from synthetic DNA sequence 150, and each of them are sequenced by sequencer 129 to determine the ID of the synthetic DNA sequence.

In embodiments, when an entire genome is designed, it is not necessary to sequence the entire genome to identify the entire DNA sequence of the genome, but only the sequence of the barcode(s). When the barcode sequences are known, the entire genome can be identified by searching a database containing the synthetic genome design. In these cases, one or multiple barcodes may be used, allowing the mixing and matching of synthetic DNA subparts to create various genome combinations.

FIG. 7 illustrates graphical user interface 160 of a synthetic DNA barcode design according to embodiments. Parameters and representative data entry fields for a barcode library are displayed at a terminal, the display of which solicits their input by a user. Basic parameters include, e.g., the name of the barcode set 161, the length of the barcode(s) 163, and total number of barcodes 164 to be designed.

The minimum number of base differences between each barcode 165 can be set. This solicitation addresses the issue of incorrect sequence identification due to sequencing error. For example, if barcode A (related to sequence A) and barcode B (related to sequence B) have only a single base difference, then when barcode B is sequenced it could be incorrectly identified as representing unrelated sequence A due to sequencing error. Minimum 166 and maximum 167 GC content can also be set, which effects sequencing efficiency and many other methods relating to the manipulation of DNA. Non-limiting examples include the efficiency of PCR amplification of DNA strands, and PCR performed specifically to prepare DNA for sequencing. A number of attempts to randomly provide acceptable barcodes 168 can also be set.

In embodiments, a terminal also displays indicia to solicit single or multiple sequence blacklists, for new input 169 or recall from storage 171 by a user. Blacklists are lists of sequences that the calculated digitally represented sequence should not include in any barcodes in order to avoid a large range of technical problems. Black list sequences may include, but are not limited to, specific restriction enzymes, primer sequences, transcription start sites or other barcode libraries.

In embodiments, once the user has input required parameters calculations are performed in one or more attempts to design a desired barcode library. If unable to design a desired barcode library a terminal displays indicia that will inform the user that a barcode could not be calculated, allowing the user to adjust parameters and attempt to design again.

In embodiments, if successful in designing the desired barcodes, instructions may cause transmission or output of the barcode sequences as digital data with or without related instructions, including but not limited to, one or more of a text file, comma-separated values (CSV) file, a database[what does this mean, specifically?], and instructions to display a request that instructions be transmitted to a networked device, that in turn causes, directly or indirectly instruct a machine that the desired segue (s) is/are to be physically synthesized.

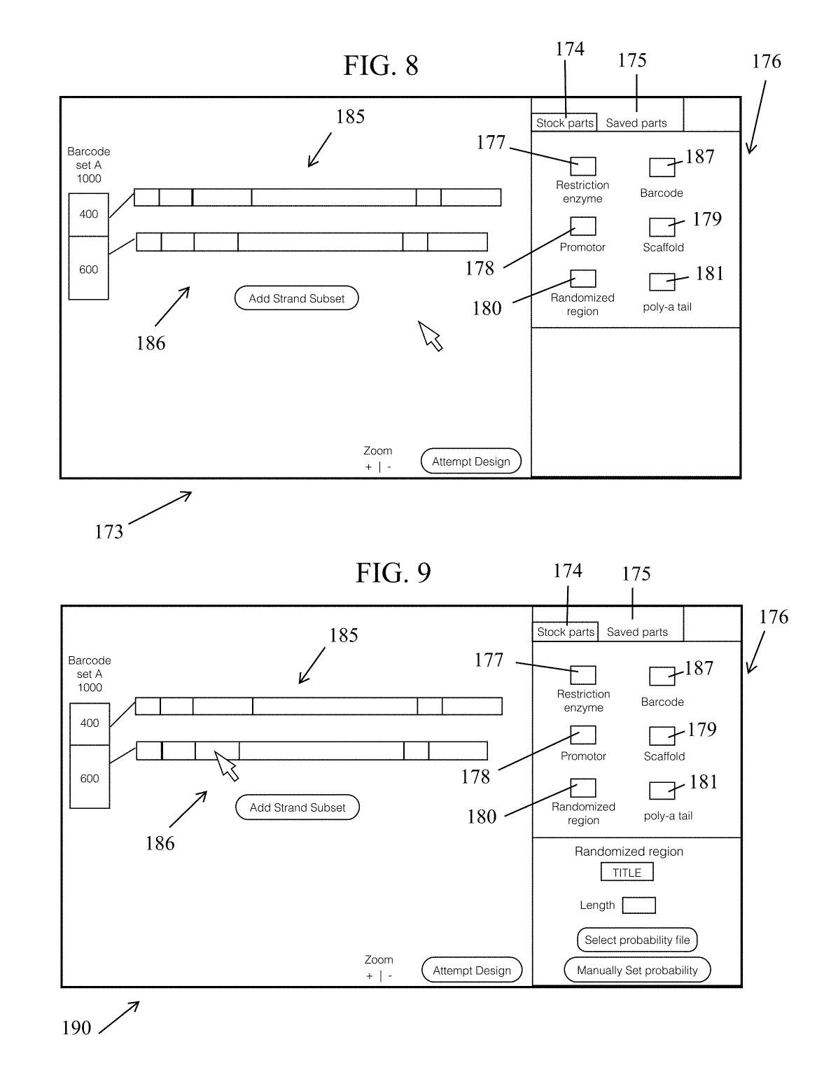

FIG. 8 illustrates a graphical user interface 173 of a synthetic DNA barcode design method according to embodiments. A terminal displays indicia that pre-determined, pre-stored, or pre-selected partial sequences of DNA may be selected by the user and connected to form linear DNA strands with the option to create randomized libraries of DNA sequences. The user can select (185 and 186) stock parts 174, and/or saved parts 175, transmitted from a remote location, or otherwise indicated at a terminal, or custom parts 176 created and input by the user. These DNA custom parts include, but are not limited to, one or more of bar code 187, restriction enzyme sites 177, promoter regions 178, poly-A tails 181, and genes. Scaffold regions 179 are DNA sequence parts that are static, and randomized regions 180 that are calculated according to a random sequence generator algorithm that randomizes within parameters set by a user.

In embodiments, a terminal displays indicia that a user can design and input data representing regions flanking the barcodes, including but not limited to, one or more of restriction enzyme sites, photo-cleavable sites, and chemical cleavable sites.

In embodiments, a terminal displays indicia calling upon the user to design scaffold regions that can be used as universal primer targets for specific amplification of barcode regions.

In embodiments, a terminal displays indicia the t user can use to select, to assign a predesigned set of barcodes to the library, or input barcode parameters to design barcode(s) and randomized library(ies) together. Synthetic DNA strands are designed as subsets within a barcode set. The number of strands to be designed in each subset can be modified by the user by a terminal displaying indicia that the user may change the number of barcodes associated with that particular subset. The user can perform this modification by inputting the exact number of barcodes associated with a strand subset, or by graphically manipulating an icon of a barcode library in order to adjust a number of barcodes associated with each strand subset. Similar to the blacklist feature described above (FIG. 7), a blacklist for a synthetic DNA designer can also be input by a user, preventing instructions to design or transmit unwanted DNA sequences.

FIG. 9 illustrates a graphical user interface of a synthetic DNA barcode design method according to embodiments. In embodiments, a terminal displays GUI 190 and indicia 191 that randomized regions can be randomized regions of DNA sequences, including but not limited to, DNA coding for amino acids, and non-encoding RNA.

In embodiments, a terminal displays indicia that a user can set minimum and maximum guanine-cytosine (GC) content parameters.

In embodiments, a terminal displays indicia that user can set the probabilities for the amino acids or DNA bases of a randomized region by selecting a file with probabilities, or by setting the probabilities within software. This will be discussed in greater detail below.

Still referring to FIG. 9, after the user has designed a desired synthetic DNA library, the instructions based on user input at a terminal will be executed in an attempt to design the library, within the parameters set by the user. If unable to design the desired library, a terminal will inform the user, allowing the user to adjust parameters and attempt design again.

In embodiments, if successful in designing the desired library, instructions may cause transmission or output of the library as digital data with or without related instructions, including but not limited to, one or more of a text file, comma-separated values (CSV) file, a database-specific file, and instructions to display a request that instructions be transmitted to a networked device, that in turn causes, directly or indirectly instruct a machine that the desired sequence(s) is/are to be physically synthesized.

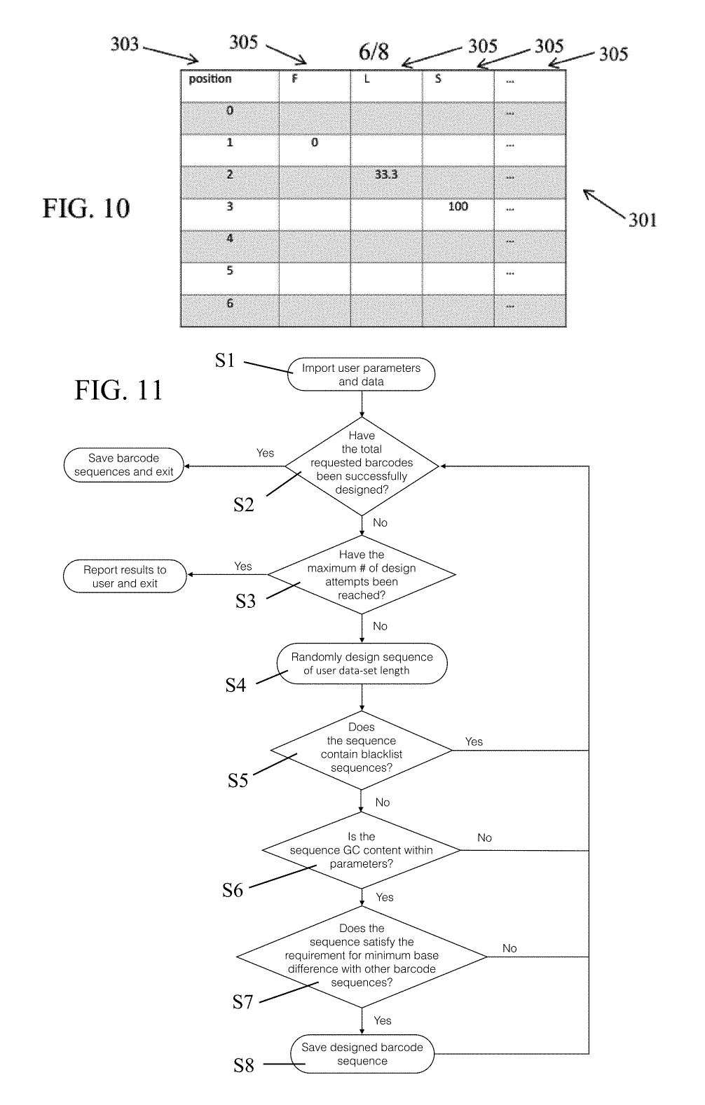

FIG. 10 illustrates an exemplary data structure (e.g., a data table) for setting probabilities for a synthetic DNA barcode design method according to embodiments. In embodiments, exemplary data structure 301 for setting probabilities is used to set the probabilities (in %) that a given amino acid (AA) will appear at a given position. The first column 303 is the position (along a 3′ to 5′ read) of the AA in the randomized region, and columns 305 represent the 20 naturally occurring AAs. The probabilities in one row should not sum to more than 100. If an AA probability is set to 100 in the matrix, then that AA will stay constant at that position in the randomized region. For example, every randomized region using the probability data structure as illustrated will have an S at position 3. Likewise, if the AA probability is set to 0, then that AA will never be used at that position. The total probabilities in a row should add up to 100. However, the probabilities for all AAs at all positions do not need to be set. In embodiments, all the AA probabilities set in that row may be summed and subtracted from 100, and the remainder are be distributed as probabilities for all the AAs in that row that were not set. For example, as illustrated in FIG. 10 at AA position 2 in the randomized region, if L has a probability of 33.3% and the other AA probabilities are not set, then all other 19 AAs have a probability (100−33.3)/19=3.5%.

FIG. 11 is a non-limiting exemplary flow diagram for an algorithm for a barcode sequence design according to embodiments. In Step 1 (S1), data representing the user parameter(s) and user data are imported. In S2, if the total number of requested barcodes are successfully designed, the barcode sequences are saved and the process is exited. If not exited, instructions are executed to determine if the maximum number of design attempts has been reached (S3). If yes, the results are reported to the user and the sub-process is exited. If no, the process continues to S4, wherein a random design sequence of user set length (indicated by user-input data) is generated. In S5, the process determines if the sequence contains blacklist sequences. If yes, the process returns to S2. If no, the process determines if the sequence GC content is within the parameters (S6). If no, the process returns to S2. If yes, the process proceeds to S7, wherein the system determines if the sequence satisfies the requirement for minimum base difference with other barcode sequences. If no, the process returns to S2. If yes, the process proceeds to Step 8, and the designed barcode sequence is saved.

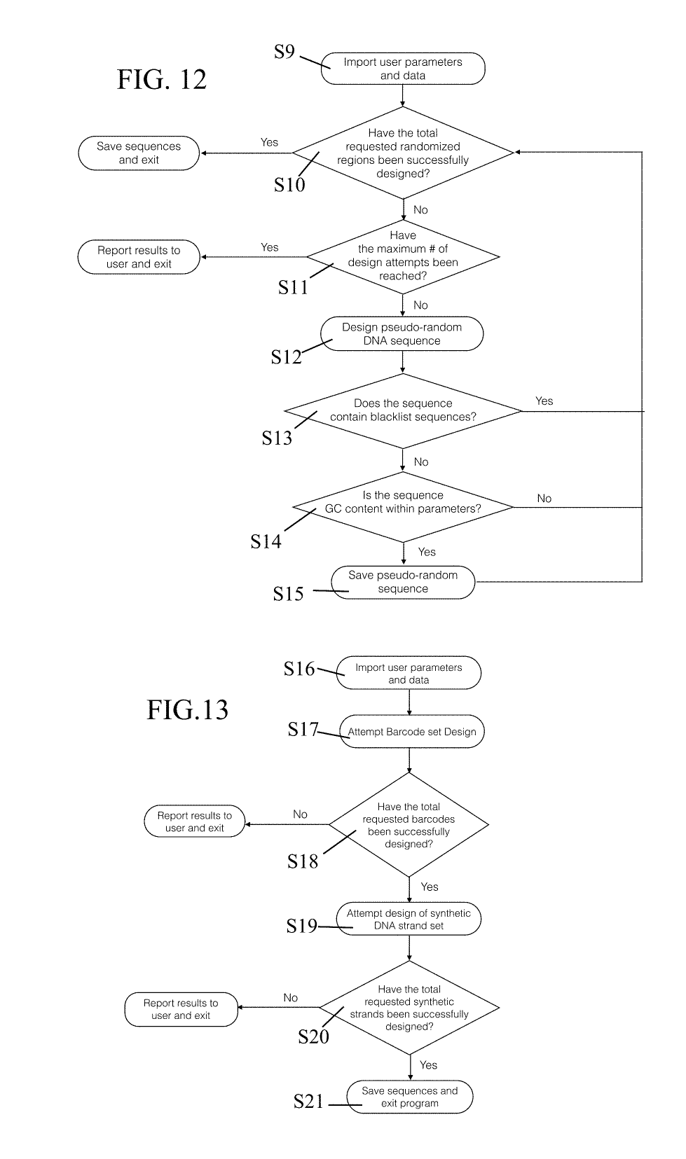

FIG. 12 is a flow diagram for an algorithm for a randomized region design according to embodiments. In S9, the user parameter and data are imported. In S10, if the total requested barcodes were successfully designed, the barcode sequences are saved and the system is exited. If no, the process determines if the maximum number of design attempts has been reached (S11). If yes, the results are reported to the user and the system is exited. If no, the process continues to S12, wherein a pseudo-random DNA sequence is designed. In S13, the process determines if the sequence contains blacklist sequences. If yes, the process returns to S10. If no, the process determines if the sequence GC content is within the parameters (S14). If no, the process returns to S10. If yes, the process proceeds to S15, wherein the pseudo-random DNA sequence is saved.

FIG. 13 is a flow diagram for an algorithm for a synthetic DNA barcode design system according to embodiments. In S16, the user parameter and data are imported. In S17, the barcode and set design is attempted. In S18, a system determines if the total requested barcodes were successfully designed. If no, the barcode sequences are saved and the system is exited. If no, the results are reported to the user and the system is exited. If yes, the process proceeds to S19, wherein a synthetic strand set design is attempted. In S20, the system determines if the total requested synthetic strands have been successfully designed. If no, the barcode sequences are saved and the system is exited. If yes, the process proceeds to S21, wherein the sequence is saved and the program is exited.

In embodiments, although a graphical user interface is shown above, a command line interface or executing a script can be used in embodiments. Along these same lines, in embodiments, a terminal can additionally or alternately communicate any of the displayed indicia or information herein, in whole or in part, or also, via audio, video, or other user-understandable broadcast method.

In embodiments, synthetic barcodes may or may not be used, for example, in synthetic DNA that codes for any one or more of the following examples: crops and/or livestock (to designate organism sourcing geography, indigenous location, growth conditions, intellectual property coverage information, growth or harvesting facility(ies), age, dates of growth and/or harvesting milestones); food stuffs; genes to designate what entity manufactured the gene.

### System and Digital Communications Network Hardware

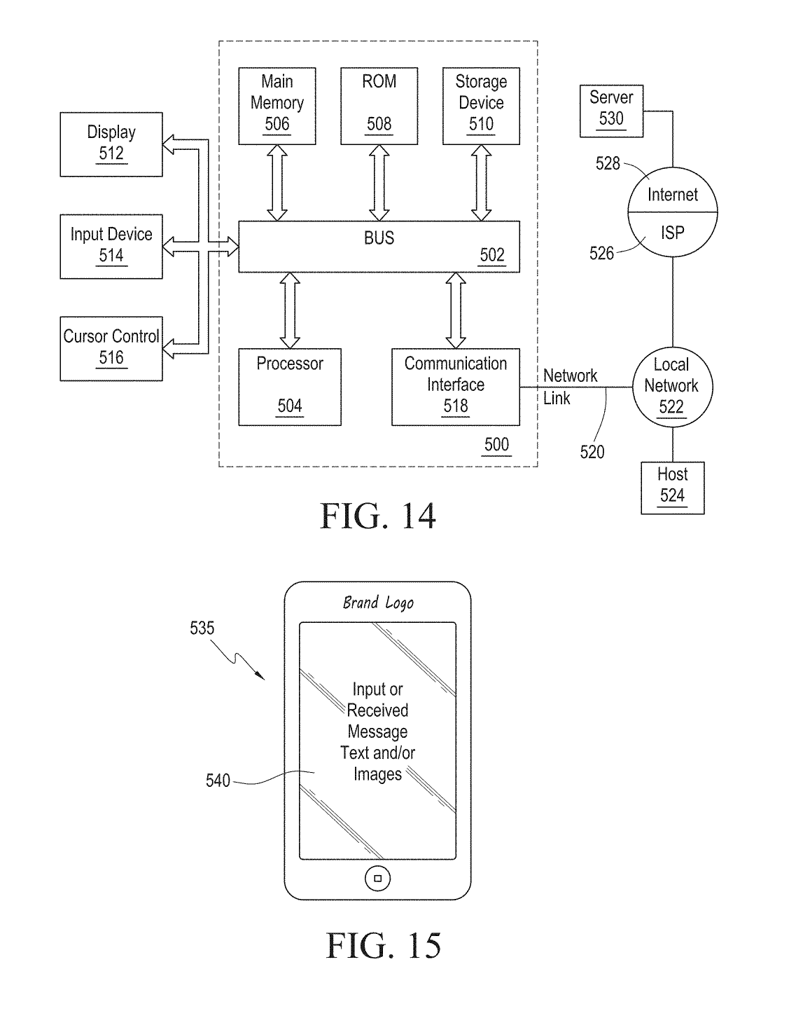

Another aspect of the disclosure is a computer system. Referring to FIG. 14 and according to embodiments, the techniques described herein are implemented by one or more special-purpose computing devices. The special-purpose computing devices may be hard-wired to perform the techniques, or may include digital electronic devices such as one or more application-specific integrated circuits (ASICs) or field programmable gate arrays (FPGAs) that are persistently programmed to perform the techniques, or may include one or more general purpose hardware processors programmed to perform the techniques pursuant to program instructions in firmware, memory, other storage, or a combination. Such special-purpose computing devices may also combine custom hard-wired logic, ASICs, or FPGAs with custom programming to accomplish the techniques. The special-purpose computing devices may be desktop computer systems, portable computer systems, handheld devices, networking devices or any other device that incorporates hard-wired and/or program logic to implement the techniques.

For example, FIG. 14 is a block diagram that illustrates a computer system 500 upon which an embodiment may be implemented. Computer system 500 includes a bus 502 or other communication mechanism for communicating information, and a hardware processor 504 coupled with bus 502 for processing information. Hardware processor 504 may be, for example, a general purpose microprocessor.

Computer system 500 also includes a main memory 506, such as a random access memory (RAM) or other dynamic storage device, coupled to bus 502 for storing information and instructions to be executed by processor 504. Main memory 506 also may be used for storing temporary variables or other intermediate information during execution of instructions to be executed by processor 504. Such instructions, when stored in non-transitory storage media accessible to processor 504, render computer system 500 into a special-purpose machine that is customized to perform the operations specified in the instructions.

Computer system 500 further includes a read only memory (ROM) 508 or other static storage device coupled to bus 502 for storing static information and instructions for processor 504. A storage device 510, such as a magnetic disk or optical disk, is provided and coupled to bus 502 for storing information and instructions.

Computer system 500 may be coupled via bus 502 to a display 512, such as a cathode ray tube (CRT), for displaying information to a computer user. An input device 514, including alphanumeric and other keys, is coupled to bus 502 for communicating information and command selections to processor 504. Another type of user input device is cursor control 516, such as a mouse, a trackball, or cursor direction keys for communicating direction information and command selections to processor 504 and for controlling cursor movement on display 512. This input device typically has two degrees of freedom in two axes, a first axis (e.g., x) and a second axis (e.g., y), that allows the device to specify positions in a plane.

Computer system 500 may implement the techniques described herein using customized hard-wired logic, one or more ASICs or FPGAs, firmware and/or program logic which in combination with the computer system causes or programs computer system 500 to be a special-purpose machine. According to at least one embodiment, the techniques herein are performed by computer system 500 in response to processor 504 executing one or more sequences of one or more instructions contained in main memory 506. Such instructions may be read into main memory 506 from another storage medium, such as storage device 510. Execution of the sequences of instructions contained in main memory 506 causes processor 504 to perform the process operations described herein. In alternative embodiments, hard-wired circuitry may be used in place of or in combination with software instructions.

The terms “storage media” and “storage device” as used herein refer to any non-transitory media that store data and/or instructions that cause a machine to operate in a specific fashion. Such storage media may comprise non-volatile media and/or volatile media. Non-volatile media includes, for example, optical or magnetic disks, such as storage device 510. Volatile media includes dynamic memory, such as main memory 506. Common forms of storage media include, for example, a floppy disk, a flexible disk, hard disk, solid state drive, magnetic tape, or any other magnetic data storage medium, a CD-ROM, any other optical data storage medium, any physical medium with patterns of holes, a RAM, a PROM, and EPROM, a FLASH-EPROM, NVRAM, any other memory chip or cartridge.

Storage media and storage device are distinct from but may be used in conjunction with transmission media. Transmission media participates in transferring information between storage media/devices. For example, transmission media includes coaxial cables, copper wire and fiber optics, including the wires that comprise bus 502. Transmission media can also take the form of acoustic or light waves, such as those generated during radio-wave and infra-red data communications.

Various forms of media may be involved in carrying one or more sequences of one or more instructions to processor 504 for execution. For example, the instructions may initially be carried on a magnetic disk or solid state drive of a remote computer. The remote computer can load the instructions into its dynamic memory and send the instructions over a telephone line using a modem. A modem local to computer system 500 can receive the data on the telephone line and use an infra-red transmitter to convert the data to an infra-red signal. An infra-red detector can receive the data carried in the infra-red signal and appropriate circuitry can place the data on bus 502. Bus 502 carries the data to main memory 506, from which processor 504 retrieves and executes the instructions. The instructions received by main memory 506 may optionally be stored on storage device 510 either before or after execution by processor 504.

Computer system 500 also includes a communication interface 518 coupled to bus 502. Communication interface 518 provides a two-way data communication coupling to a network link 520 that is connected to a local network 522. For example, communication interface 518 may be an integrated services digital network (ISDN) card, cable modem, satellite modem, or a modem to provide a data communication connection to a corresponding type of telephone line. As another example, communication interface 518 may be a local area network (LAN) card to provide a data communication connection to a compatible LAN. Wireless links may also be implemented. In at least one such implementation, communication interface 518 sends and receives one or more of electrical, electromagnetic and optical signals (as with all uses of “one or more” herein implicitly including any combination of one or more of these) that carry digital data streams representing various types of information.

Network link 520 typically provides data communication through one or more networks to other data devices. For example, network link 520 may provide a connection through local network 522 to a host computer 524 or to data equipment operated by an Internet Service Provider (ISP) 526. ISP 526 in turn provides data communication services through the world wide packet data communication network now commonly referred to as the “Internet” 528. Local network 522 and Internet 528 both use electrical, electromagnetic or optical signals that carry digital data streams. The signals through the various networks and the signals on network link 520 and through communication interface 518, which carry the digital data to and from computer system 500, are example forms of transmission media.

Computer system 500 can send messages and receive data, including program code, through the network(s), network link 520 and communication interface 518. In at least one embodiment of the Internet example, a server 530 might transmit a requested code for an application program through Internet 528, ISP 526, local network 522 and communication interface 518.

In embodiments, the received code may be one or more of executed by processor 504 as it is received, and/or stored in storage device 510, or other non-volatile storage for later execution.

Now referring to FIG. 15, in at least one embodiment a device used in accordance with this disclosure is or comprises mobile display or touch screen input smart phone or tablet 535, which is shown displaying direct user-to-device input message text and or image(s), or remotely received message text and/or image(s) 540.

### Computer-Readable Medium

Another aspect of the disclosure is one or more computer-readable media (or computer storage apparatus) having a program, which when executed by one or more processors, such part of one or more of the systems described herein, causes the one or more processors to enable, allow or cause devices to perform any one of the methods as variously comprising any one or more of its various embodiments or sub-embodiments described above or otherwise covered by the appended claims.

In embodiments, the one or more computer-readable media are non-transitory media such as, but not limited to HDD and SSD disk drives, thumb and other flash drives, DVDs, CDs, various static and dynamic storage devices and other numerous storage media.

In embodiments, the one or more computer-readable media comprise or are one or more transitory electronic signals.

The following numbered clauses set forth various embodiments of the disclosure:

1. A method, terminal, system, or transitory or non-transitory computer-readable media according to any one or more of the preceding or following clauses, comprising (optionally means for) one or more of: (a) selectively reducing data available to, or processed by one or more computers communicatively connected to, a digitally-stored DNA library database, (b) improving DNA sequence data security (c) increasing operational efficiency of one or more computers communicatively connected to one or more DNA sequencer(s), (d) reducing the demands on non-digital-computing laboratory resources, and (e) reducing error introduced during sequencing and related DNA analysis, at a terminal in a digital communications network, comprising:

(optionally means for) displaying indicia, broadcasting data, or transmitting instructions, to solicit collection of or to access data uniquely representative of, or uniquely indicating, one or more digitally-input synthetic DNA design parameters;

2. A method, terminal, system, or transitory or non-transitory computer-readable media according to any one or more of the preceding or following clauses, comprising (optionally means for) one or more of: collecting or accessing data uniquely representative of, or uniquely indicating, the one or more digitally-input synthetic DNA design parameters;

3. A method, terminal, system, or transitory or non-transitory computer-readable media according to any one or more of the preceding or following clauses, comprising (optionally means for) one or more of: transmitting data necessary to cause communication of the data uniquely representative of, or uniquely indicating, the one or more digitally-input synthetic DNA design parameters to one or more of the terminal and at least one remote computer(s);

4. A method, terminal, system, or transitory or non-transitory computer-readable media according to any one or more of the preceding or following clauses, comprising (optionally means for) one or more of: establishing direct or indirect communication access and linkage between the terminal and either (a) a store at the terminal, or (b) the at least one remote computer(s), on which are stored, or by which access is available to receive:
data representative of an entire synthetic DNA sequence of a DNA library, the synthetic DNA sequence being (remotely or at the terminal) calculated based on the one or more DNA design parameters; and
data representative of an entire synthetic DNA barcode sequence that is calculated based on the calculated synthetic DNA sequence

5. A method, terminal, system, or transitory or non-transitory computer-readable media according to any one or more of the preceding or following clauses, comprising (optionally means for) one or more of: transmitting instructions from the terminal or at least one remote computer(s) or one or more high throughput sequencer(s), to cause the one or more high throughput DNA sequencer(s) to sequence one or more of:
only the synthetic DNA barcode;
the synthetic DNA barcode;
less than one or more of 0.1%, 1%, 2%, 3%, 4%, 5%, 6%, 10%, 15%, 20%, 25%, 30%, 40%, 45%, 50%, and 60% of the entire synthetic DNA sequence; and
no portion of the synthetic DNA sequence that is part of the synthetic DNA barcode.

6. A method, terminal, system or, transitory or non-transitory computer-readable medium according to any one or more of the preceding or following clauses,
wherein the DNA barcode sequence is uniquely associated and uniquely representative of, as relative to all of the DNA sequences in the DNA library, the calculated synthetic DNA sequence.

7. A method, terminal, system or, transitory or non-transitory computer-readable medium according to any one or more of the preceding or following clauses,
wherein the instructions are calculated to cause the at least one remote computer(s) based on the data representative of an entire synthetic DNA barcode sequence that is calculated based on the synthetic DNA sequence.

8. A method, terminal, system or, transitory or non-transitory computer-readable medium according to any one or more of the preceding or following clauses,
wherein the synthetic DNA barcode is located before a transcription start region of the synthetic DNA sequence, optionally in one or more of:
   a ribosome binding site region;
   a ribosome promoter region;
   a RNA polymerase binding site;
   a start codon;
   a transcription factor binding site; and
   an enhancer region.

9. A method, terminal, system or, transitory or non-transitory computer-readable medium according to any one or more of the preceding or following clauses,
wherein the synthetic DNA barcode is an inside intron of the synthetic DNA sequence.

10. A method, terminal, system or, transitory or non-transitory computer-readable medium according to any one or more of the preceding or following clauses,
wherein the synthetic DNA barcode is located adjacent to one or more of:
   a restriction enzyme digestion site;
   a photo-cleavable site; and
   a chemical-cleavable site.

11. A method, terminal, system or, transitory or non-transitory computer-readable medium according to any one or more of the preceding or following clauses,
wherein the synthetic DNA barcode comprises at least one primer regions for polymerase chain reaction amplification.

12. A method, terminal, system or, transitory or non-transitory computer-readable medium according to any one or more of the preceding or following clauses,
wherein the synthetic DNA barcode is a subset of the synthetic DNA sequence.

13. A method, terminal, system or, transitory or non-transitory computer-readable medium according to any one or more of the preceding or following clauses, wherein the one or more synthetic DNA design parameters comprise one or more of:
at least one name of a barcode set;
at least one base-pair length of one or more barcode(s);
at least one total number of barcode(s);
at least one minimum number of different bases between barcodes;
at least one minimum number of combined guanine (G) and cytosine (C) content in the synthetic DNA sequence;
at least one maximum number of combined guanine (G) and cytosine (C) content in the synthetic DNA sequence;
at least one number of random attempts to calculate a barcode;
at least one blacklisted DNA barcode sequence;
at least one saved list of blacklisted DNA barcode sequences;
at least one restriction enzyme sequence;
at least one promoter sequence;
at least one scaffold sequence;
at least one randomized sequence region;
at least one poly-A-tail;
at least one stop codon; and
at least one graphically displayed synthetic DNA sequence subset order.

14. A method, terminal, system or, transitory or non-transitory computer-readable medium according to any one or more of the preceding or following clauses,
wherein the synthetic DNA design parameters comprise at least one probability by percentage, that an amino acid will being selected at a given sequence position when calculating a randomized sequence region.

15. A method, terminal, system or, transitory or non-transitory computer-readable medium according to any one or more of the preceding or following clauses, comprising (optionally means for):
calculating, or receiving data based on at least a calculation of, variations of amino acid region(s) based on the one or more digitally-input synthetic DNA design parameters.

16. A method, terminal, system or, transitory or non-transitory computer-readable medium according to any one or more of the preceding or following clauses, comprising (optionally means for):
calculating, or receiving data based on at least a calculation of, a random synthetic DNA sequence that codes for one or more amino acids of sufficient number and position on the DNA sequence to code for one or more of at least one peptide and at least one protein.

17. A method, terminal, system or, transitory or non-transitory computer-readable medium according to any one or more of the preceding or following clauses,
wherein the barcode identifies both, in the same sequence and by the same single nucleotide coding region (optionally within a same single region of a single strand of the synthetic DNA), DNA that within the same nucleotides functions as (1) RNA-coding DNA that can be translated into protein and (2) mRNA-coding DNA wherein the mRNA has one or more secondary structures of (a) duplex(es); (b) single-stranded region(s); (c) bulge(s); (d) internal loop(s) or any combination thereof, and optionally one or more of (a), (b), (c) and (d) in combination with one or more hairpin structure(s).

18. A method, terminal, system or, transitory or non-transitory computer-readable medium according to any one or more of the preceding or following clauses,
wherein the DNA library comprises DNA that encodes for the group consisting of one or more of:
   nucleic acid not having self-complementing regions sufficient to form a hairpin loop;
   nucleic acid not having internal homology sufficient to form a duplex;
   non-hairpin RNA;
   entire organs;
   entire biological systems;
   entire organisms;
   entire tissue types; and
   entire proteins.

19. A method, terminal, system or, transitory or non-transitory computer-readable medium according to any one or more of the preceding or following clauses,
wherein the DNA library comprises DNA that encodes for the group comprising one or more of:
   nucleic acid not having self-complementing regions sufficient to form a hairpin loop;
   nucleic acid not having internal homology sufficient to form a duplex;
   non-hairpin RNA;
   entire organs;
   entire biological systems;
   entire organisms;
   entire tissue types; and
   entire proteins.

20. A method, terminal, system or, transitory or non-transitory computer-readable medium according to any one or more of the preceding or following clauses,
wherein the synthetic DNA sequence consists of one or more of;
   a purely synthetic DNA polynucleotide;
   a purely synthetic DNA oligonucleotide;
   a collection of purely synthetic DNA polynucleotides;
   a collection of purely synthetic DNA oligonucleotides;
   a synthetic DNA polynucleotide;
   a synthetic DNA oligonucleotide;
   a collection of synthetic DNA polynucleotides;
   a collection of synthetic DNA oligonucleotides; and
   any combination thereof.

21. A method, terminal, system or, transitory or non-transitory computer-readable medium according to any one or more of the preceding or following clauses,
wherein the synthetic DNA sequence consists essentially of one or more of;
   a purely synthetic DNA polynucleotide;
   a purely synthetic DNA oligonucleotide;
   a collection of purely synthetic DNA polynucleotides;
   a collection of purely synthetic DNA oligonucleotides;
   a synthetic DNA polynucleotide;
   a synthetic DNA oligonucleotide;
   a collection of synthetic DNA polynucleotides;
   a collection of synthetic DNA oligonucleotides; and
   any combination thereof.

22. A method, terminal, system or, transitory or non-transitory computer-readable medium according to any one or more of the preceding or following clauses,
wherein the synthetic DNA sequence comprises one or more of;
   a purely synthetic DNA polynucleotide;
   a purely synthetic DNA oligonucleotide;
   a collection of purely synthetic DNA polynucleotides;
   a collection of purely synthetic DNA oligonucleotides;
   a synthetic DNA polynucleotide;
   a synthetic DNA oligonucleotide;
   a collection of synthetic DNA polynucleotides;
   a collection of synthetic DNA oligonucleotides; and
   any combination thereof.

Embodiments can work with software, hardware, and/or operating system implementations other than those described herein. Any software, hardware, and operating system implementations suitable for performing the functions described herein can be used. Embodiments are applicable to both a client and to a server or a combination of both.

While it is apparent that the illustrative embodiments of the disclosure herein fulfil one or more objectives or inventive solutions, it is appreciated that numerous modifications and other embodiments may be devised by those skilled in the art. Additionally, one or more feature(s), sub-feature(s), micro-feature(s), element(s), sub-element(s) and/or microelement(s) from any embodiment may be used singly or in combination with one or more of any feature(s), sub-feature(s), micro-feature(s), element(s), sub-element(s) and/or microelement(s) from any same or other embodiment(s). Therefore, it will be understood that the appended claims are intended to cover all such modifications and embodiments that would come within the spirit and scope of the present disclosure.

1. A method of one or more of (a) selectively reducing data available to, or processed by one or more computers communicatively connected to, a digitally-stored DNA library database, (b) improving DNA sequence data security (c) increasing operational efficiency of one or more computers communicatively connected to one or more DNA sequencer(s), (d) reducing the demands on non-digital-computing laboratory resources, and (e) reducing error introduced during sequencing and related DNA analysis, at a terminal in a digital communications network, comprising:
displaying indicia, broadcasting data, or transmitting instructions, to solicit collection of or to access data uniquely representative of, or uniquely indicating, one or more digitally-input synthetic DNA design parameters;
collecting or accessing data uniquely representative of, or uniquely indicating, the one or more digitally-input synthetic DNA design parameters;
transmitting data necessary to cause communication of the data uniquely representative of, or uniquely indicating, the one or more digitally-input synthetic DNA design parameters to one or more of:
a store at the terminal and
at least one remote computer(s);
establishing direct or indirect communication access and linkage between the terminal and one or more of:
(a) the store at the terminal, and
(b) the at least one remote computer(s);
on which is/are stored, or by which access is available to receive:
data representative of an entire synthetic DNA sequence of a DNA library, the synthetic DNA sequence being (remotely or at the terminal) calculated based on the one or more DNA design parameters; and
data representative of an entire synthetic DNA barcode sequence that is calculated based on the calculated synthetic DNA sequence;
wherein the DNA barcode sequence is uniquely associated and uniquely representative of, as relative to all of the DNA sequences in the DNA library, the calculated synthetic DNA sequence; and
wherein the DNA library comprises DNA that encodes for the group consisting of one or more of:
nucleic acid not having self-complementing regions sufficient to form a hairpin loop;
nucleic acid not having internal homology sufficient to form a duplex;
non-hairpin RNA;
entire organs;
entire biological systems;
entire organisms;
entire tissue types; and
entire proteins.

2-26. (canceled)
